(19) United States
(12) Patent Application Publication
(10) Pub. No.: US 2024/0424018 A1
(43) Pub. Date: Dec. 26, 2024

(54) USING SILVER COMPOUNDS TO PROTECT BLOOD CELLS

(71) Applicant: SilverCeuticals Inc., Lindon, UT (US)

(72) Inventors: Keith W. Moeller, Highland, UT (US); Cameron D. Moeller, Pleasant Grove, UT (US); Kirk W. Moeller, American Fork, UT (US); Kory R. Dopp, Eagle Mountain, UT (US); Bracken J. Moeller, Lehi, UT (US); Kimball K. Moeller, American Fork, UT (US); Brennan R. Moeller, Highland, UT (US)

(21) Appl. No.: 18/339,864

(22) Filed: Jun. 22, 2023

(51) Int. Cl. A61K 33/38; A61K 9/00

(52) U.S. Cl. CPC A61K 33/38; A61K 9/0053

(57) ABSTRACT

Compounds and methods for protecting blood cells of a subject are disclosed. One method includes providing a compound including silver particles to a subject and facilitating daily ingestion of the compound by the subject. The compound is provided to the subject and facilitating daily ingestion of the compound by the subject is performed to protect blood cells in the subject. The blood cells can be protected during chemotherapy and may include red blood cells, white blood cells, or both red blood cells and white blood cells.

### FIELD

The subject matter disclosed herein relates to silver compounds and, more particularly, relates to methods for using silver compounds to protect blood cells.

### BACKGROUND

Chemotherapy is one technique for treating cancer. When a subject (e.g., a human, animal, etc.) is exposed to the chemical substances used in chemotherapy (e.g., a cytotoxic and/or other drug(s)), the subject's red blood cells and/or white blood cells are often killed and/or at least damaged along with the target cancerous cells, which can result in a reduced red blood cell count and/or white blood cell count for the subject. A reduced red blood cell count and/or reduced white blood cell count can adversely affect the subject's health and/or overall well-being during chemotherapy and/or the subject's ability to recover from chemotherapy, potentially leaving the subject vulnerable to a number of issues.

### BRIEF SUMMARY

The subject matter of the present disclosure provides examples of silver compounds and corresponding methods that use silver compounds to protect blood cells. There is a desire to utilize silver compounds to protect red blood cells and/or white blood cells, particularly during chemotherapy. It would therefore be desirable to develop improved methods for using silver compounds to protect red blood cells and/or white blood cells, which can increase or at least maintain the red blood cell count and/or white blood cell count of a subject (e.g., a human, animal, etc.) prior to starting chemotherapy and/or during chemotherapy. Accordingly, the subject matter of the present application has been developed in response to the present state of the art, and in particular, in response to the damage caused to red blood cells and/or white blood cells of a subject during chemotherapy.

Disclosed herein is a method for using silver compounds to protect blood cells of a subject (e.g., the subject's red blood cells and/or white blood cells). The method includes providing a compound comprising silver (Ag) particles to a subject and facilitating daily ingestion of the compound by the subject in which providing the compound to the subject and facilitating daily ingestion of the compound by the subject is performed to protect blood cells in the subject. The preceding subject matter of this paragraph characterizes example 1 of the present disclosure.

The compound comprises 0.5 ppm to 100 ppm of the Ag particles. The preceding subject matter of this paragraph characterizes example 2 of the present disclosure, wherein example 2 also includes the subject matter according to example 1, above.

The Ag particles comprise one of nano-silver particles and colloidal silver particles. The preceding subject matter of this paragraph characterizes example 3 of the present disclosure, wherein example 3 also includes the subject matter according to any one of examples 1 or 2, above.

Facilitating daily ingestion of the compound by the subject comprises facilitating daily ingestion of an amount of the compound in the range of 0.15 ounces to 15 ounces. The preceding subject matter of this paragraph characterizes example 4 of the present disclosure, wherein example 4 also includes the subject matter according to any one of examples 1 or 2, above.

Facilitating daily ingestion of the compound by the subject comprises facilitating daily Q ingestion of an amount of the compound in the range of 0.15 ounces to 8 ounces. The preceding subject matter of this paragraph characterizes example 5 of the present disclosure, wherein example 5 also includes the subject matter according to any one of examples 1 or 2, above.

Facilitating daily ingestion of the compound by the subject comprises facilitating daily ingestion of 8 ounces of the compound. The preceding subject matter of this paragraph characterizes example 6 of the present disclosure, wherein example 6 also includes the subject matter according to any one of examples 1 or 2, above.

Facilitating daily ingestion of the compound by the subject comprises facilitating ingestion of the compound two times daily. The preceding subject matter of this paragraph characterizes example 7 of the present disclosure, wherein example 7 also includes the subject matter according to any one of examples 1 or 2, above.

Facilitating ingestion of the compound two times daily comprises facilitating ingestion of 3 ounces or 4 ounces of the compound two times daily. The preceding subject matter of this paragraph characterizes example 8 of the present disclosure, wherein example 8 also includes the subject matter according to any one of examples 1, 2, or 7, above.

The two times daily are separated by a predetermined minimum amount of time. The preceding subject matter of this paragraph characterizes example 9 of the present disclosure, wherein example 9 also includes the subject matter according to any one of examples 1, 2, 7, or 8, above.

Providing the compound to the subject and facilitating ingestion of the compound by the subject is performed in response to the subject preparing for chemotherapy. The preceding subject matter of this paragraph characterizes example 10 of the present disclosure, wherein example 10 also includes the subject matter according to example 1, above.

Providing the compound to the subject and facilitating ingestion of the compound by the subject is further performed in response to the subject currently receiving chemotherapy. The preceding subject matter of this paragraph characterizes example 11 of the present disclosure, wherein example 11 also includes the subject matter according to any one of examples 1 or 10, above.

Providing the compound to the subject and facilitating ingestion of the compound by the subject is performed in response to the subject currently receiving chemotherapy. The preceding subject matter of this paragraph characterizes example 12 of the present disclosure, wherein example 12 also includes the subject matter according to example 1, above.

Protecting the blood cells in the subject comprises at least one of raising and maintaining a count of blood cells in the subject. The preceding subject matter of this paragraph characterizes example 13 of the present disclosure, wherein example 13 also includes the subject matter according to example 1, above.

The count of blood cells in the subject comprises a count of white blood cells. The preceding subject matter of this paragraph characterizes example 14 of the present disclosure, wherein example 14 also includes the subject matter according to any one of examples 1 or 13, above.

The count of blood cells in the subject comprises a count of red blood cells. The preceding subject matter of this paragraph characterizes example 15 of the present disclosure, wherein example 15 also includes the subject matter according to any one of examples 1 or 13, above.

The count of blood cells in the subject comprises a first count of white blood cells and a second count of red blood cells. The preceding subject matter of this paragraph characterizes example 16 of the present disclosure, wherein example 16 also includes the subject matter according to any one of examples 1 or 13, above.

The Ag particles comprise one of nano-silver particles and colloidal silver particles. The preceding subject matter of this paragraph characterizes example 17 of the present disclosure, wherein example 17 also includes the subject matter according to example 1, above.

The compound comprises 10 ppm of the Ag particles. The preceding subject matter of this paragraph characterizes example 18 of the present disclosure, wherein example 18 also includes the subject matter according to example 1, above.

The compound comprises 32 ppm of the Ag particles. The preceding subject matter of this paragraph characterizes example 19 of the present disclosure, wherein example 19 also includes the subject matter according to example 1, above.

The compound comprises one of 10 ppm and 32 ppm of the Ag particles and the Ag particles comprise nano-silver particles. The preceding subject matter of this paragraph characterizes example 20 of the present disclosure, wherein example 20 also includes the subject matter according to example 1, above.

### BRIEF DESCRIPTION OF THE DRAWINGS

A more particular description of the embodiments briefly described above will be rendered by reference to specific embodiments that are illustrated in the appended drawings. Understanding that these drawings depict only some embodiments and are not therefore to be considered to be limiting of scope, the embodiments will be described and explained with additional specificity and detail through the use of the accompanying drawings, in which:

FIG. 1 is a schematic diagram of one embodiment of a silver particle that can protect blood cells of a subject;

FIG. 2 is a schematic diagram of one embodiment of a molecule of coating for the silver particle illustrated in FIG. 1;

FIGS. 3 through 6 are schematic flow diagrams of various embodiments of a method for using a silver compound to protect blood cells of a subject;

FIG. 7 is a schematic flow diagram of one embodiment of a method for increasing the red blood cells and/or white blood cells of a subject before chemotherapy;

FIG. 8 is a schematic flow diagram of one embodiment of a method for increasing the red blood cells and/or white blood cells of a subject during chemotherapy;

FIG. 9 is a schematic flow diagram of one embodiment of a method for maintaining the red blood cells and/or white blood cells of a subject during chemotherapy;

FIG. 10 is a schematic flow diagram of one embodiment of a method for increasing the red blood cells and/or white blood cells of a subject prior to and during chemotherapy; and

FIG. 11 is a schematic flow diagram of one embodiment of a method for increasing the red blood cells and/or white blood cells of a subject prior to chemotherapy and maintaining the increased red blood cells and/or the increased white blood cells during chemotherapy.

### DETAILED DESCRIPTION

The various embodiments disclosed herein provide silver compounds and methods that can protect blood cells (e.g., red blood cells and/or white blood cells). The silver compounds include silver particles that, when ingested by a subject (e.g., a human, an animal, etc.), can protect the subject's blood cells.

As used herein the terms, “protect blood cells,” can include, but are not limited to, increasing the number/quantity of red blood cells and/or a subject's red blood cell count, maintaining the number/quantity of red blood cells and/or a subject's red blood cell count, increasing the number/quantity of white blood cells and/or a subject's white blood cell count, and/or maintaining the number/quantity of white blood cells and/or a subject's white blood cell count. In certain embodiments, the terms, “protect blood cells,” can include protecting the red blood cells and/or white blood cells of a subject before the subject receives chemotherapy and/or while the subject is receiving chemotherapy.

In addition, “while receiving chemotherapy,” “participating in chemotherapy,” “during chemotherapy,” and the like terms can include, but are not limited to, the period of time beginning when a subject is exposed to an initial dose of the chemical substances used in chemotherapy (e.g., a cytotoxic and/or other drug(s)) and ending when the subject is exposed to a final dose of the chemical substances used in chemotherapy and/or a time subsequent to the subject's exposure to the final dose of the chemical substances used in chemotherapy. Further, “preparing to begin chemotherapy,” “prior to receiving chemotherapy,” “before receiving chemotherapy,” “before chemotherapy,” and the like terms can include, but are not limited to, a period of time ending when a subject is exposed to an initial dose of the chemical substances used in chemotherapy.

In various embodiments, the subject's blood cells are protected by increasing the subject's red blood cell count and/or increasing the subject's white blood cell count prior to the subject receiving chemotherapy. In additional or alternative embodiments, the subject's blood cells are protected by increasing the subject's red blood cell count and/or increasing the subject's white blood cell count while the subject is receiving chemotherapy. In further additional or alternative embodiments, the subject's blood cells are protected by increasing the subject's red blood cell count and/or increasing the subject's white blood cell count prior to the subject receiving chemotherapy and further increasing the subject's red blood cell count and/or further increasing the subject's white blood cell while the subject is receiving chemotherapy.

In other additional or alternative embodiments, the subject's blood cells are protected by increasing the subject's red blood cell count and/or increasing the subject's white blood cell count prior to the subject receiving chemotherapy and maintaining the subject's increased red blood cell count and/or maintaining the subject's increased white blood cell while the subject receiving chemotherapy. In further additional or alternative embodiments, the subject's blood cells are protected by maintaining the subject's red blood cell count and/or maintaining the subject's white blood cell while the subject is receiving chemotherapy.

As disclosed herein, “silver particles” can include, but are not limited to, “nano-silver particles,” “nano-silver,” “colloidal silver particles,” “colloidal silver,” “ionic silver particles,” “ionic silver,” “solution silver particles,” “solution silver,” “suspension silver particles,” and “suspension silver,” among other particles that can include silver that are possible and contemplated herein. In some embodiments, “silver particles” refers to particles that, in whole or in part, comprise silver in an aqueous medium or solution.

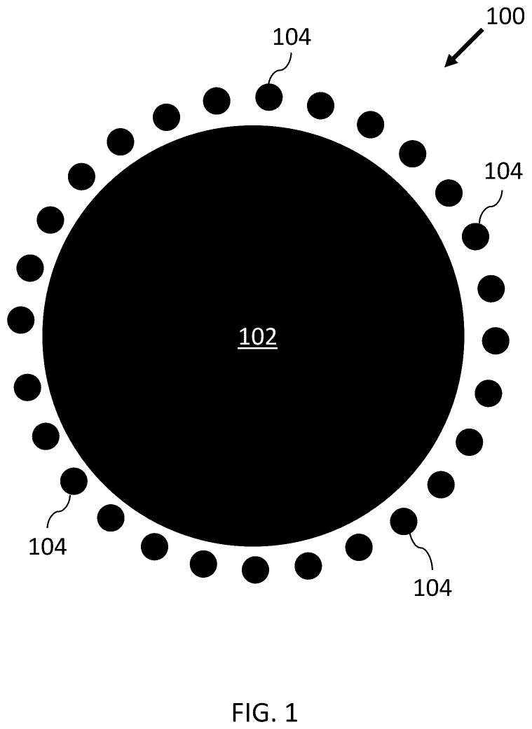

With reference to the drawings, FIG. 1 is a diagram of one embodiment of a silver particle 100, which can also be referred to herein as, an Ag particle. A silver particle 100 may be characterized in terms of valence of the silver included therein. In the exemplary embodiment shown in FIG. 1, the silver particle 100 includes a silver core 102 and a silver coating 104.

A silver core 102, in various embodiments, includes metallic silver (Ag(0)) to form a metallic silver core. In additional embodiments, the silver core 102 includes metallic nano-silver to form a metallic nano-silver core.

In various embodiments, the silver coating 104 comprises Ag(I), Ag(II), or Ag(III) in an ionic oxidation state. In some embodiments, the silver coating 104 includes silver oxide (e.g., AgO). That is, the silver particle 100 comprises Ag(0) and AgO, where AgO is present as a coating on the silver core 102 (e.g., a metallic nano-silver core).

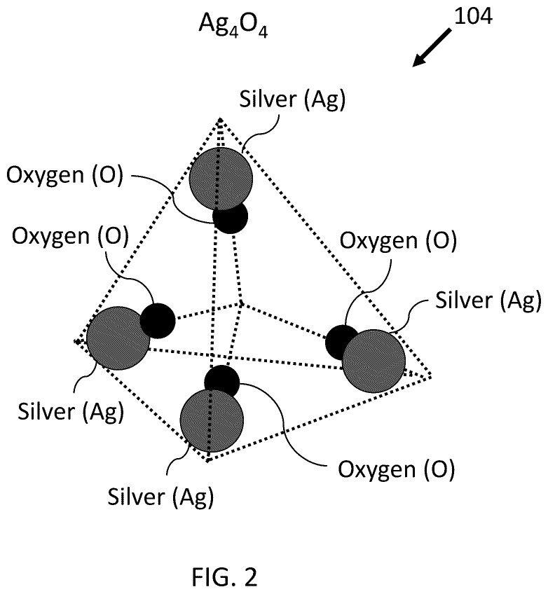

In other embodiments (see, FIG. 2), the silver coating 104 may be at least partially in the form of silver II oxide (Ag₄O₄). In a molecule of this material, two of the silver atoms are in the 1+ state (silver I) and the other two silver molecules are in the 3+ state (silver III). Under certain conditions, these molecules can give rise to silver atoms (Ag) in the 2+(silver II) state attached to respective oxygen atoms (O). Thus, the present disclosure provides silver particles comprising a metallic silver core 102 and silver coating 104 of silver oxide, the silver oxide being selected from AgO and Ag₄O₄.

The purity of a silver particle 100 may be any suitable percentage of silver purity that is capable of disinfecting a surface. In various embodiments, the percentage of silver purity of a silver particle 100 is in the range of about 50% to about 99.9999%, among other percentages of silver purity that are greater than 99.9999% or less than 50% that are possible and contemplated herein. In certain embodiments, the percentage of silver purity of a silver particle 100 is about 99.99%, among other percentages of silver purity that are greater than or less than about 99.99% that are possible and contemplated herein.

In various embodiments, a plurality of silver particles 100 are added to and/or mixed with one or more liquids and/or solutions to form a silver compound and/or silver composition that can be used to protect the red blood cells and/or white blood cells of a subject. In certain embodiments, the silver compound and/or silver composition can be used to increase or at least maintain the number of red blood cells (e.g., increase or maintain the red blood cell count) and/or increase or at least maintain the number of white blood cells (e.g., increase or maintain the white blood cell count) in a subject. The silver particles 100 can be added to and/or mixed with any suitable liquid that can facilitate, enable, and/or allow the silver particles 100 to protect the red blood cells and/or white blood cells of a subject.

In various embodiments, one liquid includes water (H₂O) and/or other aqueous solution. In some embodiments, the water or aqueous solution includes pure water and/or purified water.

An aqueous compound comprising silver particles 100 may be prepared using any suitable method and/or technique that is known or developed in the future. For example, the preparation of a compound comprising silver particles 100 may utilize an electrochemical cell comprising electrodes. One process includes: (a) placing a silver electrode in contact with a quantity of high purity water; (b) conveying electrical current through the silver electrode to thereby separate particles of silver from the silver electrode in a manner sufficient to cause production of suspended silver particles within the water; and (c) agitating the water during said production of suspended silver particles to thereby disperse the silver particles into a more uniform concentration within the water such that a higher quantity of silver particles can be produced per batch.

Another example technique for preparing a compound comprising silver particles 100 includes: (a) establishing an electrical circuit comprising a current source, and a first conductor electrically connected to the current source and a second conductor electrically connected to the current source in which the first conductor is spaced apart from the second conductor, and at least one of the conductors is made of elemental silver; (b) closing the circuit by placing the first conductor and the second conductor in communication with a fluidic resistor; (c) operating the current source to supply alternating current simultaneously to the first conductor and the second conductor such that voltage is increasing and decreasing within the first and second conductors in alternating tandem to thereby cause silver particles to separate from the first electrode and enter the fluidic resistor and become disposed in suspension within the fluidic resistor; and (d) selectively adjusting the electrodes by moving them toward the fluidic resistor to compensate for decrease in electrode length due to gradual separation of silver particles therefrom to thereby prevent arcing from occurring between the electrodes and said fluidic resistor.

In various embodiments, the total amount of silver particles 100 in a silver compound that can protect a subject's red blood cells and/or white blood cells is in the range of about 0.5 parts per million (0.5 ppm) to about 100 parts per million (100 ppm), among other quantities of silver particles 100 that are less than 0.5 ppm and greater than 100 ppm that are possible, each of which is contemplated herein. In some embodiments, the total amount of silver particles in a compound is about 10 ppm, among other amounts of silver particles 100 greater than or less that about 10 ppm that are possible, each of which is contemplated herein. In other embodiments, the total amount of silver particles in a compound is about 32 ppm, among other amounts of silver particles 100 greater than or less that about 32 ppm that are possible, each of which is contemplated herein.

As the silver particles 100 become smaller, a given concentration of silver particles 100 will represent a larger quantity of particles 100. In addition, the total surface area for a given particle concentration will increase. Therefore, particle size and/or range of particle size for the silver particles 100 may further characterize the compounds of the various embodiments, which may include any suitable particle size and/or range of particle size that is/are known or developed in the future capable of protecting a subject's blood cells (e.g., the subject's red blood cells and/or white blood cells).

In some embodiments, a silver compound comprises 50-100% by weight and/or volume of the silver particles 100 including a maximum dimension that is less than about 1.0 micrometer (1000 nanometers (nm)), among other percentages and sizes that are possible, each of which is contemplated herein. In certain embodiments, a silver compound comprises 50-100% by weight and/or volume of the silver particles 100 including a dimension in the range of about 0.005 micrometers (5 nm) to about 0.01 micrometers (10 nm), among other percentages and sizes that are possible, each of which is contemplated herein. In still other embodiments, a silver compound comprises 50-100% by weight and/or volume of the silver particles 100 including a minimum dimension greater than or equal to about 0.001 micrometers (1 nm), among other percentages and sizes that are possible, each of which is contemplated herein. Here, these silver particles 100 can be considered colloidal silver particles.

In further embodiments, a silver compound comprises 50-100% by weight and/or volume of the silver particles 100 including a maximum dimension that is less than about 0.001 micrometers (1 nm), among other percentages and sizes that are possible, each of which is contemplated herein. Here, these silver particles 100 can be considered ionic silver particles or solution silver particles.

In still further embodiments, a silver compound comprises 50-100% by weight and/or volume of the silver particles 100 including a minimum dimension that is greater than about 1.0 micrometer (1000 nm), among other percentages and sizes that are possible, each of which is contemplated herein. Here, these silver particles 100 can be considered suspension silver particles.

In one embodiment, the silver particles 100 are stable in essentially and/or substantially pure water that is free of, for example, surfactants, etc., among other natural impurities and/or unnatural impurities that are possible and contemplated herein. Additionally, or in another embodiment, the compound of silver particles 100 is essentially and/or substantially colorless.

In various embodiments, the silver compound forms a silver sol. A silver sol, in various embodiments, includes a stable dispersion of solid silver particles 100 homogeneously dispersed in a liquid. In certain embodiments, the silver sol includes a stable dispersion of solid silver particles 100 homogeneously dispersed in water (e.g., pure water) and/or an aqueous solution.

Testing of the various embodiments of a silver compound disclosed herein shows that ingesting (e.g., oral ingestion) the various embodiments of a silver compound disclosed herein (e.g., on a daily basis) can increase the number of red blood cells (e.g., red blood cell count) and/or can increase the number of white blood cells (e.g., white blood cell count) in a subject before the subject receives chemotherapy and/or while the subject is receiving chemotherapy. In addition, testing of the various embodiments of a silver compound disclosed herein shows that ingesting (e.g., oral ingestion) the various embodiments of a silver compound disclosed herein (e.g., on a daily basis) while a subject is receiving chemotherapy can maintain the number of red blood cells (e.g., red blood cell count) and/or can maintain the number of white blood cells (e.g., white blood cell count) in the subject while the subject is receiving chemotherapy.

Further testing further shows that increasing the number of red blood cells (e.g., increasing the red blood cell count) and/or the number of white blood cells (e.g., increasing the white blood cell count) in a subject before the subject receives chemotherapy and/or while the subject is receiving chemotherapy can prevent and/or at least reduce the adverse effects (e.g., side effects) experienced by a subject during chemotherapy. Additional testing shows that increasing the number of red blood cells (e.g., increasing the red blood cell count) and/or the number of white blood cells (e.g., increasing the white blood cell count) in a subject before the subject receives chemotherapy and maintaining the subject's increased number red blood cells (e.g., the increased red blood cell count) and/or increased number of white blood cells (e.g., the increased white blood cell count) while the subject is receiving chemotherapy can prevent and/or at least reduce the adverse effects (e.g., side effects) experienced by the subject during chemotherapy. Other testing shows that maintaining the number red blood cells (e.g., the red blood cell count) and/or the number of white blood cells (e.g., the white blood cell count) in a subject while the subject is receiving chemotherapy can prevent and/or at least reduce the adverse effects (e.g., side effects) experienced by the subject during chemotherapy.

In addition to preventing and/or at least reducing one or more adverse effects (e.g., one or more side effects) experienced by a subject during chemotherapy, tests using a silver compound to protect the subject's red blood cells and/or white blood cells during chemotherapy shows that ingestion of the silver compound before and/or after chemotherapy does not interfere with the efficacy of chemotherapy. That is, daily ingestion of a silver compound before and/or while receiving chemotherapy to protect the subject's red blood cells and/or white blood cells during chemotherapy treatment does not adversely affect the ability of chemotherapy to eliminate bad cells (e.g., cancerous cells) in the subject.

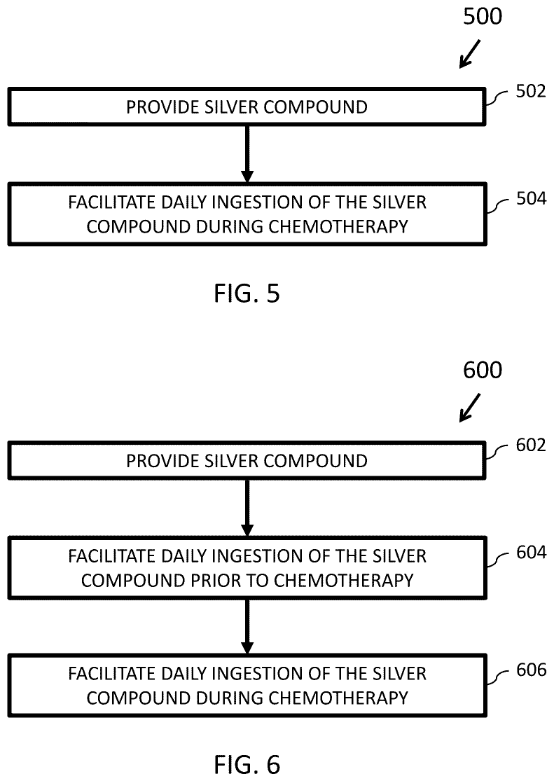

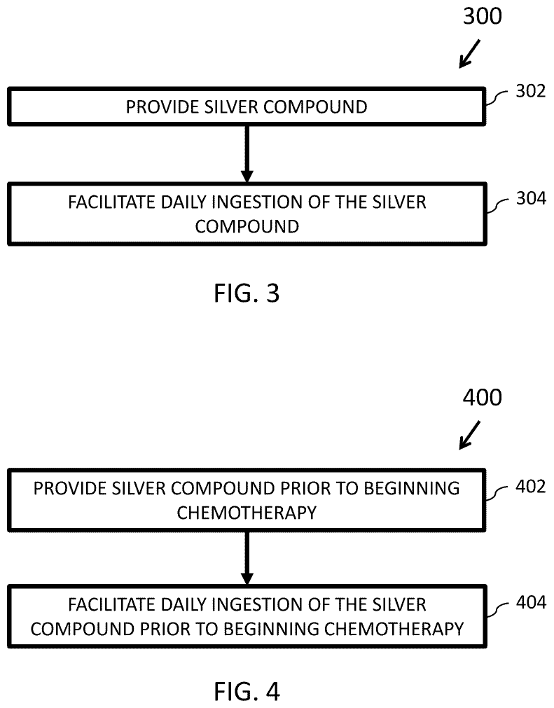

With reference to FIG. 3, FIG. 3 is a block diagram of one embodiment of a method 300 for using a silver compound to protect blood cells of a subject (e.g., the subject's red and/or white blood cells). At least in the illustrated embodiment, the method 300 can begin by a providing a silver compound to the subject (block 302).

The silver compound can be provided to the subject in any suitable manner that is known or developed in the future. Examples of providing the silver compound to the user can include, but is not limited to, by prescription, over the counter, an online purchase, and/or a subscription, etc., among other techniques/methods that are possible, each of which is contemplated herein.

The silver compound can include any embodiment of the silver particles 100 discussed herein. That is, the silver particles 100 may include nano-silver particles 100, colloidal silver particles 100, ionic silver particles 100, solution silver particles 100, and/or suspension silver particles 100.

In some embodiments, the silver compound includes an amount of silver particles 100 (e.g., Ag particles), which can include any amount of silver particles 100 in the range of about 0.5 ppm to about 100 ppm, as discussed elsewhere herein. In certain embodiments, the amount of silver particles 100 is 10 ppm or 32 ppm, among other amounts that are possible, each of which is contemplated herein.

The method 300 further includes facilitating daily ingestion of the silver compound including the silver particles 100 by the subject (block 304). Facilitating daily ingestion of the compound including the silver particles 100 may include any suitable technique and/or method that encourages and/or causes a subject to ingest the silver compound including the silver particles 100 on a daily and/or other similar regular basis. Examples of facilitating daily ingestion of the compound including the silver particles 100 can include, but are not limited to, by prescription, via instructions, by an advertisement, via a study, by a written article (e.g., online, journal, text, etc.), and/or via a recommendation (e.g., by a manufacturer, a healthcare professional, etc.), etc., among other similar techniques and/or methods that are possible, each of which is contemplated herein.

The silver compound including the silver particles 100 may be ingested in any suitable manner that is known or developed in the future. In various embodiments, the silver compound including the silver particles 100 is ingested orally, nasally, and/or intravenously (IV), among other techniques that are possible, each of which is contemplated herein.

In various embodiments, providing and facilitating daily ingestion of the silver compound including the silver particles 100 is performed in response to the subject preparing to begin chemotherapy. In additional or alternative embodiments, providing and facilitating daily ingestion of the silver compound including the silver particles 100 is performed in response to the subject beginning chemotherapy. In further additional or alternative embodiments, providing and facilitating daily ingestion of the silver compound including the silver particles 100 is performed in response to the subject preparing to begin chemotherapy and beginning chemotherapy.

In further additional or alternative embodiments, providing and facilitating daily ingestion of the silver compound including the silver particles 100 is performed in response to a desire to protect the subject's blood cells during chemotherapy. In some embodiments, the desire to protect the subject's blood cells during chemotherapy includes increasing the subject's red blood cell count and/or white blood cell count prior to the subject beginning chemotherapy. In additional or alternative embodiments, the desire to protect the subject's blood cells during chemotherapy includes increasing the subject's red blood cell count and/or white blood cell count while the subject is receiving chemotherapy. In further additional or alternative embodiments, the desire to protect the subject's blood cells during chemotherapy includes increasing the subject's red blood cell count and/or white blood cell count prior to chemotherapy and maintaining the increased red blood cell count and/or maintaining the increased white blood cell count during chemotherapy. In still further additional or alternative embodiments, the desire to protect the subject's blood cells during chemotherapy includes increasing the subject's red blood cell count and/or white blood cell count prior to chemotherapy and further increasing the subject's red blood cell count and/or further increasing the subject's white blood cell count during chemotherapy. In yet further additional or alternative embodiments, the desire to protect the subject's blood cells during chemotherapy includes maintaining the subject's red blood cell count and/or white blood cell count during chemotherapy.

Facilitating daily ingestion of the silver compound including the silver particles 100, in various embodiments, may include facilitating daily ingestion of any suitable amount of the silver compound capable of protecting a subject's blood cells. In some embodiments, facilitating daily ingestion can include facilitating daily ingestion of an amount of the silver compound in the range of about 0.15 ounces (0.15 oz) to about fifteen ounces (15 oz) of the silver compound, among other amounts and/or ranges of amounts that are less than 0.15 oz or greater than 15 oz that are possible, each of which is contemplated herein. In certain embodiments, facilitating daily ingestion can include facilitating daily ingestion of eight ounces (8 oz) or up to about 8 oz of the silver compound, among other amounts that are less than 8 oz or greater than 8 oz that are possible, each of which is contemplated herein.

In various embodiments, facilitating daily ingestion of the silver compound including the silver particles 100 may include ingesting the silver compound including the silver particles 100 any suitable number of times a day. For example, ingesting an amount of the silver compound including the silver particles 100 can include ingesting the silver compound once a day, two times a day, three times a day, etc., among other quantities that are possible, each of which is contemplated herein.

In at least some embodiments in which the silver compound including the silver particles 100 is ingested at least twice daily, each ingestion is separated by a predetermined minimum amount of time, which can be any suitable amount of time that can facilitating protecting the blood cells of the subject. Example predetermined minimum amounts of time can include, but are not limited to, one hour, two hours, four hours, six hours, eight hours, twelve hours, etc., among other minimum amounts of time that are possible, each of which is contemplated herein.

Referring to FIG. 4, FIG. 4 is a block diagram of another embodiment of a method 400 for using a silver compound to protect blood cells of a subject (e.g., the subject's red and/or white blood cells). At least in the illustrated embodiment, the method 400 can begin by a providing a silver compound to the subject prior to the subject beginning chemotherapy (block 402).

The silver compound can be provided to the subject in any suitable manner that is known or developed in the future. Examples of providing the silver compound to the user can include, but is not limited to, by prescription, over the counter, an online purchase, and/or a subscription, etc., among other techniques/methods that are possible, each of which is contemplated herein.

The silver compound can include any embodiment of the silver particles 100 discuss elsewhere herein. That is, the silver particles 100 may include nano-silver particles 100, colloidal silver particles 100, ionic silver particles 100, solution silver particles 100, and/or suspension silver particles 100.

In some embodiments, the silver compound includes an amount of silver particles 100, which can include any amount of silver particles 100 in the range of about 0.5 ppm to about 100 ppm, as discussed elsewhere herein. In certain embodiments, the amount of silver particles 100 is 10 ppm or 32 ppm, among other amounts that are possible, each of which is contemplated herein.

The method 400 further includes facilitating daily ingestion of the silver compound including the silver particles 100 by the subject prior to the subject receiving chemotherapy (block 404). The subject can begin daily ingestion of the silver compound including the silver particles 100 for any suitable period of time prior to beginning chemotherapy that can increase the subject's red blood count and/or white blood cell count to a predetermined desired amount/level prior to beginning chemotherapy, which can include any suitable amount/level that is known or developed in the future. In various embodiments, the subject begins daily ingestion of the silver compound including the silver particles 100 in the range of about one day to about sixty days prior to beginning chemotherapy, among other amounts of time and/or ranges of time that are possible, each of which is contemplated herein. In certain embodiments, the subject begins daily ingestion of the silver compound including the silver particles 100 fourteen days, at least fourteen days, thirty days, or at least thirty days prior to beginning chemotherapy, among other amounts of time greater than thirty days and/or less than fourteen days that are possible, each of which is contemplated herein.

Facilitating daily ingestion of the compound including the silver particles 100 prior to receiving chemotherapy may include any suitable technique and/or method that encourages and/or causes a subject to ingest the silver compound including the silver particles 100 on a daily and/or other similar regular basis. Examples of facilitating daily ingestion of the compound including the silver particles 100 can include, but are not limited to, by prescription, via instructions, by an advertisement, via a study, by a written article (e.g., online, journal, text, etc.), and/or via a recommendation (e.g., by a manufacturer, a healthcare professional, etc.), etc., among other similar techniques and/or methods that are possible, each of which is contemplated herein.

The silver compound including the silver particles 100 may be ingested in any suitable manner that is known or developed in the future. In various embodiments, the silver compound including the silver particles 100 is ingested orally, nasally, and/or intravenously (IV), among other techniques that are possible, each of which is contemplated herein.

In various embodiments, providing and facilitating daily ingestion of the silver compound including the silver particles 100 is performed in response to the subject preparing to begin chemotherapy. In additional or alternative embodiments, providing and facilitating daily ingestion of the silver compound including the silver particles 100 prior to receiving chemotherapy is performed in response to a desire to protect the subject's blood cells during chemotherapy.

In some embodiments, the desire to protect the subject's blood cells during chemotherapy includes increasing the subject's red blood cell count and/or white blood cell count prior to the subject beginning chemotherapy. In additional or alternative embodiments, the desire to protect the subject's blood cells during chemotherapy includes maintaining the subject's red blood cell count and/or white blood cell count during chemotherapy.

Facilitating daily ingestion of the silver compound including the silver particles 100 prior to chemotherapy, in various embodiments, may include facilitating daily ingestion of any suitable amount of the silver compound capable of protecting a subject's blood cells. In some embodiments, facilitating daily ingestion can include facilitating daily ingestion of an amount of the silver compound in the range of about 0.15 oz to about 15 oz of the silver compound, among other amounts and/or ranges of amounts that are less than 0.15 oz or greater than 15 oz that are possible, each of which is contemplated herein. In certain embodiments, facilitating daily ingestion can include facilitating daily ingestion of 8 oz or up to about 8 oz of the silver compound, among other amounts that are less than 8 oz or greater than 8 oz that are possible, each of which is contemplated herein.

In various embodiments, facilitating daily ingestion of the silver compound including the silver particles 100 prior to chemotherapy may include ingesting the silver compound including the silver particles 100 any suitable number of times a day. For example, ingesting an amount of the silver compound including the silver particles 100 can include ingesting the silver compound once a day, two times a day, three times a day, etc. prior to receiving chemotherapy, among other quantities that are possible, each of which is contemplated herein.

In at least some embodiments in which the silver compound including the silver particles 100 is ingested at least twice daily, each ingestion is separated by a predetermined minimum amount of time, which can be any suitable amount of time that can facilitating protecting the blood cells of the subject. Example predetermined minimum amounts of time can include, but are not limited to, one hour, two hours, four hours, six hours, eight hours, twelve hours, etc., among other minimum amounts of time that are possible, each of which is contemplated herein.

With reference to FIG. 5, FIG. 5 is a block diagram of a further embodiment of a method 500 for using a silver compound to protect blood cells of a subject (e.g., the subject's red and/or white blood cells). At least in the illustrated embodiment, the method 500 can begin by a providing a silver compound to the subject (block 502).

The silver compound can be provided to the subject before and/or after the subject begins chemotherapy. Further, the silver compound can be provided to the subject in any suitable manner that is known or developed in the future, as discussed elsewhere herein.

The silver compound can include any embodiment of the silver particles 100 discuss elsewhere herein. That is, the silver particles 100 may include nano-silver particles 100, colloidal silver particles 100, ionic silver particles 100, solution silver particles 100, and/or suspension silver particles 100.

In some embodiments, the silver compound includes an amount of silver particles 100, which can include any amount of silver particles 100 in the range of about 0.5 ppm to about 100 ppm, as discussed elsewhere herein. In certain embodiments, the amount of silver particles 100 is 10 ppm or 32 ppm, among other amounts that are possible, each of which is contemplated herein.

The method 500 further includes facilitating daily ingestion of the silver compound including the silver particles 100 by the subject while the subject is receiving chemotherapy (block 504). The subject can begin daily ingestion of the silver compound including the silver particles 100 at any point in time while the subject is receiving chemotherapy. In some embodiments, the subject begins daily ingestion of the silver compound including the silver particles 100 on the initial day of chemotherapy. In other embodiments, the subject begins daily ingestion of the silver compound including the silver particles 100 on any suitable day subsequent to the initial day of chemotherapy.

Facilitating daily ingestion of the compound including the silver particles 100 while receiving chemotherapy may include any suitable technique and/or method that encourages and/or causes a subject to ingest the silver compound including the silver particles 100 on a daily and/or other similar regular basis. Examples of facilitating daily ingestion of the compound including the silver particles 100 can include, but are not limited to, by prescription, via instructions, by an advertisement, via a study, by a written article (e.g., online, journal, text, etc.), and/or via a recommendation (e.g., by a manufacturer, a healthcare professional, etc.), etc., among other similar techniques and/or methods that are possible, each of which is contemplated herein.

The silver compound including the silver particles 100 may be ingested in any suitable manner that is known or developed in the future. In various embodiments, the silver compound including the silver particles 100 is ingested orally, nasally, and/or intravenously (IV), among other techniques that are possible, each of which is contemplated herein.

In various embodiments, providing and facilitating daily ingestion of the silver compound including the silver particles 100 is performed in response to the subject beginning chemotherapy. In additional or alternative embodiments, providing and facilitating daily ingestion of the silver compound including the silver particles 100 while receiving chemotherapy is performed in response to a desire to protect the subject's blood cells during chemotherapy.

In further additional or alternative embodiments, the desire to protect the subject's blood cells during chemotherapy includes increasing the subject's red blood cell count and/or white blood cell count while the subject is receiving chemotherapy. In still further additional or alternative embodiments, the desire to protect the subject's blood cells during chemotherapy includes maintaining the subject's red blood cell count and/or white blood cell count during chemotherapy.

Facilitating daily ingestion of the silver compound including the silver particles 100 while receiving chemotherapy, in various embodiments, may include facilitating daily ingestion of any suitable amount of the silver compound capable of protecting a subject's blood cells. In some embodiments, facilitating daily ingestion can include facilitating daily ingestion of an amount of the silver compound in the range of about 0.15 oz to about 15 oz of the silver compound, among other amounts and/or ranges of amounts that are less than 0.15 oz or greater than 15 oz that are possible, each of which is contemplated herein. In certain embodiments, facilitating daily ingestion can include facilitating daily ingestion of 8 oz or up to about 8 oz of the silver compound, among other amounts that are less than 8 oz or greater than 8 oz that are possible, each of which is contemplated herein.

In various embodiments, facilitating daily ingestion of the silver compound including the silver particles 100 prior to chemotherapy may include ingesting the silver compound including the silver particles 100 any suitable number of times a day. For example, ingesting an amount of the silver compound including the silver particles 100 can include ingesting the silver compound once a day, two times a day, three times a day, etc. prior to receiving chemotherapy, among other quantities that are possible, each of which is contemplated herein.

In at least some embodiments in which the silver compound including the silver particles 100 is ingested at least twice daily, each ingestion is separated by a predetermined minimum amount of time, which can be any suitable amount of time that can facilitating protecting the blood cells of the subject. Example predetermined minimum amounts of time can include, but are not limited to, one hour, two hours, four hours, six hours, eight hours, twelve hours, etc., among other minimum amounts of time that are possible, each of which is contemplated herein.

Referring to FIG. 6, FIG. 6 is a block diagram of a further embodiment of a method 600 for using a silver compound to protect blood cells of a subject (e.g., the subject's red and/or white blood cells). At least in the illustrated embodiment, the method 600 can begin by a providing a silver compound to the subject (block 602).

The silver compound can be provided to the subject before and/or after the subject begins chemotherapy. Further, the silver compound can be provided to the subject in any suitable manner that is known or developed in the future, as discussed elsewhere herein.

The silver compound can include any embodiment of the silver particles 100 discuss elsewhere herein. That is, the silver particles 100 may include nano-silver particles 100, colloidal silver particles 100, ionic silver particles 100, solution silver particles 100, and/or suspension silver particles 100.

In some embodiments, the silver compound includes an amount of silver particles 100, which can include any amount of silver particles 100 in the range of about 0.5 ppm to about 100 ppm, as discussed elsewhere herein. In certain embodiments, the amount of silver particles 100 is 10 ppm or 32 ppm, among other amounts that are possible, each of which is contemplated herein.

The method 600 further includes facilitating daily ingestion of the silver compound including the silver particles 100 by the subject prior to the subject receiving chemotherapy (block 604). The method 600 also includes facilitating daily ingestion of the silver compound while the subject receiving chemotherapy (block 606).

The subject can begin daily ingestion of the silver compound including the silver particles 100 for any suitable period of time prior to beginning chemotherapy that can increase the subject's red blood count and/or white blood cell count to a predetermined desired amount/level prior to beginning chemotherapy, which can include any suitable amount/level that is known or developed in the future. In various embodiments, the subject begins daily ingestion of the silver compound including the silver particles 100 in the range of about one day to about sixty days prior to beginning chemotherapy, among other amounts of time and/or ranges of time that are possible, each of which is contemplated herein. In certain embodiments, the subject begins daily ingestion of the silver compound including the silver particles 100 fourteen days, at least fourteen days, thirty days, or at least thirty days prior to beginning chemotherapy, among other amounts of time greater than thirty days and/or less than fourteen days that are possible, each of which is contemplated herein.

Facilitating daily ingestion of the compound including the silver particles 100 prior to and while receiving chemotherapy may include any suitable technique and/or method that encourages and/or causes a subject to ingest the silver compound including the silver particles 100 on a daily and/or other similar regular basis. Examples of facilitating daily ingestion of the compound including the silver particles 100 prior to and while receiving chemotherapy can include, but are not limited to, by prescription, via instructions, by an advertisement, via a study, by a written article (e.g., online, journal, text, etc.), and/or via a recommendation (e.g., by a manufacturer, a healthcare professional, etc.), etc., among other similar techniques and/or methods that are possible, each of which is contemplated herein.

The silver compound including the silver particles 100 may be ingested in any suitable manner that is known or developed in the future. In various embodiments, the silver compound including the silver particles 100 is ingested orally, nasally, and/or intravenously (IV), among other techniques that are possible, each of which is contemplated herein.

In various embodiments, providing and facilitating daily ingestion of the silver compound including the silver particles 100 prior to receiving chemotherapy is performed in response to the subject preparing to begin chemotherapy. In additional or alternative embodiments, providing and facilitating daily ingestion of the silver compound including the silver particles 100 prior to and/or while receiving chemotherapy receiving chemotherapy is performed in response to a desire to protect the subject's blood cells during chemotherapy.

In further additional or alternative embodiments, providing and facilitating daily ingestion of the silver compound including the silver particles 100 prior to and while receiving chemotherapy is performed in response to a desire to protect the subject's blood cells during chemotherapy. In some embodiments, the desire to protect the subject's blood cells during chemotherapy includes increasing the subject's red blood cell count and/or white blood cell count prior to the subject beginning chemotherapy. In additional or alternative embodiments, the desire to protect the subject's blood cells during chemotherapy includes increasing the subject's red blood cell count and/or white blood cell count while the subject is receiving chemotherapy. In further additional or alternative embodiments, the desire to protect the subject's blood cells during chemotherapy includes increasing the subject's red blood cell count and/or white blood cell count prior to chemotherapy and maintaining the increased red blood cell count and/or maintaining the increased white blood cell count during chemotherapy. In still further additional or alternative embodiments, the desire to protect the subject's blood cells during chemotherapy includes increasing the subject's red blood cell count and/or white blood cell count prior to chemotherapy and further increasing the subject's red blood cell count and/or further increasing the subject's white blood cell count during chemotherapy. In yet further additional or alternative embodiments, the desire to protect the subject's blood cells during chemotherapy includes maintaining the subject's red blood cell count and/or white blood cell count during chemotherapy.

Facilitating daily ingestion of the silver compound including the silver particles 100 prior to and/or while receiving chemotherapy, in various embodiments, may include facilitating daily ingestion of any suitable amount of the silver compound capable of protecting a subject's blood cells. In some embodiments, facilitating daily ingestion prior to and while receiving chemotherapy can include facilitating daily ingestion of an amount of the silver compound in the range of about 0.15 oz to about 15 oz of the silver compound, among other amounts and/or ranges of amounts that are less than 0.15 oz or greater than 15 oz that are possible, each of which is contemplated herein. In certain embodiments, facilitating daily ingestion prior to and while receiving chemotherapy can include facilitating daily ingestion of 8 oz or up to about 8 oz of the silver compound, among other amounts that are less than 8 oz or greater than 8 oz that are possible, each of which is contemplated herein. The same or different amounts can be used prior to and while receiving chemotherapy.

In various embodiments, facilitating daily ingestion of the silver compound including the silver particles 100 prior to and/or while receiving chemotherapy may include ingesting the silver compound including the silver particles 100 any suitable number of times a day. For example, ingesting an amount of the silver compound including the silver particles 100 can include ingesting the silver compound once a day, two times a day, three times a day, etc. prior to receiving chemotherapy, among other quantities that are possible, each of which is contemplated herein. The same or different frequency/frequencies can be used prior to and while receiving chemotherapy.

In at least some embodiments in which the silver compound including the silver particles 100 is ingested at least twice daily, each ingestion is separated by a predetermined minimum amount of time, which can be any suitable amount of time that can facilitating protecting the blood cells of the subject. Example predetermined minimum amounts of time can include, but are not limited to, one hour, two hours, four hours, six hours, eight hours, twelve hours, etc., among other minimum amounts of time that are possible, each of which is contemplated herein.

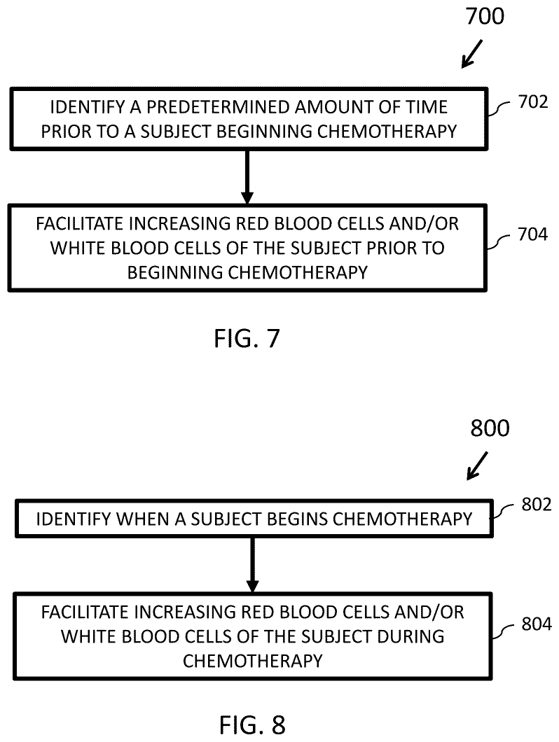

With reference to FIG. 7, FIG. 7 is a block diagram of one embodiment of a method 700 for increasing the red blood cells (e.g., red blood cell count) and/or white blood cells (e.g., white blood cell count) of a subject before chemotherapy. At least in the illustrated embodiment, the method 700 can begin by identifying a predetermined amount of time prior to a subject beginning chemotherapy (block 702).

The predetermined amount of time prior to the subject beginning chemotherapy can include any suitable amount of time prior to beginning chemotherapy that can provide sufficient time to increase the subject's red blood count and/or white blood cell count to a predetermined desired amount/level prior to beginning chemotherapy, which can include any suitable amount/level that is known or developed in the future. In various embodiments, the predetermined amount of time prior to the subject beginning chemotherapy is in the range of about one day to about sixty days prior to beginning chemotherapy, among other amounts of time and/or ranges of time that are possible, each of which is contemplated herein. In certain embodiments, the predetermined amount of time prior to the subject beginning chemotherapy is fourteen days, at least fourteen days, thirty days, or at least thirty days prior to beginning chemotherapy, among other amounts of time greater than thirty days and/or less than fourteen days that are possible, each of which is contemplated herein.

The method 700 further includes facilitating increasing the red blood cells (e.g., increase the red blood cell count) and/or white blood cells (e.g., increase the white blood cell count) of the subject prior to the subject beginning chemotherapy (block 704). In various embodiments, a silver compound including any of the various embodiments of the silver particles 100 discussed herein can facilitate increasing the red blood cells and/or white blood cells of the subject prior to the subject beginning chemotherapy.

In some embodiments, facilitating increasing the red blood cells and/or white blood cells of the subject prior to the subject beginning chemotherapy includes providing a silver compound including silver particles 100 to the subject (see, e.g., block 402 discussed with reference to FIG. 4). The silver compound can be provided to the subject in any suitable manner that is known or developed in the future. Examples of providing the silver compound to the user can include, but is not limited to, by prescription, over the counter, an online purchase, and/or a subscription, etc., among other techniques/methods that are possible, each of which is contemplated herein.

The silver compound can include any embodiment of the silver particles 100 discuss elsewhere herein. That is, the silver particles 100 may include nano-silver particles 100, colloidal silver particles 100, ionic silver particles 100, solution silver particles 100, and/or suspension silver particles 100.

In some embodiments, the silver compound includes an amount of silver particles 100, which can include any amount of silver particles 100 in the range of about 0.5 ppm to about 100 ppm, as discussed elsewhere herein. In certain embodiments, the amount of silver particles 100 is 10 ppm or 32 ppm, among other amounts that are possible, each of which is contemplated herein.

In additional or alternative embodiments, facilitating increasing the red blood cells and/or white blood cells of the subject prior to the subject beginning chemotherapy includes facilitating daily ingestion of the silver compound including the silver particles 100 by the subject prior to the subject receiving chemotherapy (see, e.g., block 404 discussed with reference to FIG. 4).

The subject can begin daily ingestion of the silver compound including the silver particles 100 for any suitable period of time prior to beginning chemotherapy that can increase the subject's red blood count and/or white blood cell count to a predetermined desired amount/level prior to beginning chemotherapy, which can include any suitable amount/level that is known or developed in the future. In various embodiments, the subject begins daily ingestion of the silver compound including the silver particles 100 in the range of about one day to about sixty days prior to beginning chemotherapy, among other amounts of time and/or ranges of time that are possible, each of which is contemplated herein. In certain embodiments, the subject begins daily ingestion of the silver compound including the silver particles 100 fourteen days, at least fourteen days, thirty days, or at least thirty days prior to beginning chemotherapy, among other amounts of time greater than thirty days and/or less than fourteen days that are possible, each of which is contemplated herein.

Facilitating daily ingestion of the compound including the silver particles 100 prior to receiving chemotherapy may include any suitable technique and/or method that encourages and/or causes a subject to ingest the silver compound including the silver particles 100 on a daily and/or other similar regular basis. Examples of facilitating daily ingestion of the compound including the silver particles 100 can include, but are not limited to, by prescription, via instructions, by an advertisement, via a study, by a written article (e.g., online, journal, text, etc.), and/or via a recommendation (e.g., by a manufacturer, a healthcare professional, etc.), etc., among other similar techniques and/or methods that are possible, each of which is contemplated herein.

The silver compound including the silver particles 100 may be ingested in any suitable manner that is known or developed in the future. In various embodiments, the silver compound including the silver particles 100 is ingested orally, nasally, and/or intravenously (IV), among other techniques that are possible, each of which is contemplated herein.

In various embodiments, providing and/or facilitating daily ingestion of the silver compound including the silver particles 100 is performed in response to the subject preparing to begin chemotherapy. In additional or alternative embodiments, providing and/or facilitating daily ingestion of the silver compound including the silver particles 100 prior to receiving chemotherapy is performed in response to a desire to protect the subject's blood cells during chemotherapy.

Facilitating daily ingestion of the silver compound including the silver particles 100 prior to chemotherapy, in various embodiments, may include facilitating daily ingestion of any suitable amount of the silver compound capable of protecting a subject's blood cells. In some embodiments, facilitating daily ingestion can include facilitating daily ingestion of an amount of the silver compound in the range of about 0.15 oz to about 15 oz of the silver compound, among other amounts and/or ranges of amounts that are less than 0.15 oz or greater than 15 oz that are possible, each of which is contemplated herein. In certain embodiments, facilitating daily ingestion can include facilitating daily ingestion of 8 oz or up to about 8 oz of the silver compound, among other amounts that are less than 8 oz or greater than 8 oz that are possible, each of which is contemplated herein.

In various embodiments, facilitating daily ingestion of the silver compound including the silver particles 100 prior to chemotherapy may include ingesting the silver compound including the silver particles 100 any suitable number of times a day. For example, ingesting an amount of the silver compound including the silver particles 100 can include ingesting the silver compound once a day, two times a day, three times a day, etc. prior to receiving chemotherapy, among other quantities that are possible, each of which is contemplated herein.

In at least some embodiments in which the silver compound including the silver particles 100 is ingested at least twice daily, each ingestion is separated by a predetermined minimum amount of time, which can be any suitable amount of time that can facilitating protecting the blood cells of the subject. Example predetermined minimum amounts of time can include, but are not limited to, one hour, two hours, four hours, six hours, eight hours, twelve hours, etc., among other minimum amounts of time that are possible, each of which is contemplated herein.

Referring to FIG. 8, FIG. 8 is a block diagram of one embodiment of a method 800 for increasing the red blood cells (e.g., red blood cell count) and/or white blood cells (e.g., white blood cell count) during chemotherapy. At least in the illustrated embodiment, the method 800 can begin by identifying a time when a subject begins chemotherapy (block 802).

A subject begins chemotherapy when the subject is exposed to an initial dose of the chemical substances used in chemotherapy (e.g., a cytotoxic and/or other drug(s)). A subject ends chemotherapy when the subject is exposed to a final dose of the chemical substances used in chemotherapy and/or includes a time subsequent to the subject's exposure to the final dose of the chemical substances used in chemotherapy.

The method 800 further includes facilitating increasing the red blood cells (e.g., increase the red blood cell count) and/or white blood cells (e.g., increase the white blood cell count) of the subject during chemotherapy (block 804). In various embodiments, a silver compound including any of the various embodiments of the silver particles 100 discussed herein can facilitate increasing the red blood cells and/or white blood cells of the subject during chemotherapy.

In some embodiments, facilitating increasing the red blood cells (e.g., increase the red blood cell count) and/or white blood cells (e.g., increase the white blood cell count) of the subject during chemotherapy includes providing a silver compound to the subject (see, e.g., block 502 discussed with reference to FIG. 5). The silver compound can be provided to the subject before and/or after the subject begins chemotherapy. Further, the silver compound can be provided to the subject in any suitable manner that is known or developed in the future, as discussed elsewhere herein.

The silver compound can include any embodiment of the silver particles 100 discuss elsewhere herein. That is, the silver particles 100 may include nano-silver particles 100, colloidal silver particles 100, ionic silver particles 100, solution silver particles 100, and/or suspension silver particles 100.

In some embodiments, the silver compound includes an amount of silver particles 100, which can include any amount of silver particles 100 in the range of about 0.5 ppm to about 100 ppm, as discussed elsewhere herein. In certain embodiments, the amount of silver particles 100 is 10 ppm or 32 ppm, among other amounts that are possible, each of which is contemplated herein.

In additional or alternative embodiments, facilitating increasing the red blood cells (e.g., increase the red blood cell count) and/or white blood cells (e.g., increase the white blood cell count) of the subject during chemotherapy includes facilitating daily ingestion of the silver compound including the silver particles 100 by the subject during chemotherapy (see, e.g., block 504 discussed with reference to FIG. 5). The subject can begin daily ingestion of the silver compound including the silver particles 100 at any point in time while the subject is receiving chemotherapy.

In some embodiments, the subject begins daily ingestion of the silver compound including the silver particles 100 on the initial day of chemotherapy. In other embodiments, the subject begins daily ingestion of the silver compound including the silver particles 100 on any suitable day subsequent to the initial day of chemotherapy.

Facilitating daily ingestion of the compound including the silver particles 100 while receiving chemotherapy may include any suitable technique and/or method that encourages and/or causes a subject to ingest the silver compound including the silver particles 100 on a daily and/or other similar regular basis. Examples of facilitating daily ingestion of the compound including the silver particles 100 can include, but are not limited to, by prescription, via instructions, by an advertisement, via a study, by a written article (e.g., online, journal, text, etc.), and/or via a recommendation (e.g., by a manufacturer, a healthcare professional, etc.), etc., among other similar techniques and/or methods that are possible, each of which is contemplated herein.

The silver compound including the silver particles 100 may be ingested in any suitable manner that is known or developed in the future. In various embodiments, the silver compound including the silver particles 100 is ingested orally, nasally, and/or intravenously (IV), among other techniques that are possible, each of which is contemplated herein.

In various embodiments, providing and/or facilitating daily ingestion of the silver compound including the silver particles 100 is performed in response to the subject beginning chemotherapy. In additional or alternative embodiments, providing and/or facilitating daily ingestion of the silver compound including the silver particles 100 while receiving chemotherapy is performed in response to a desire to protect the subject's blood cells during chemotherapy.

Facilitating daily ingestion of the silver compound including the silver particles 100 while receiving chemotherapy, in various embodiments, may include facilitating daily ingestion of any suitable amount of the silver compound capable of protecting a subject's blood cells. In some embodiments, facilitating daily ingestion can include facilitating daily ingestion of an amount of the silver compound in the range of about 0.15 oz to about 15 oz of the silver compound, among other amounts and/or ranges of amounts that are less than 0.15 oz or greater than 15 oz that are possible, each of which is contemplated herein. In certain embodiments, facilitating daily ingestion can include facilitating daily ingestion of 8 oz or up to about 8 oz of the silver compound, among other amounts that are less than 8 oz or greater than 8 oz that are possible, each of which is contemplated herein.

In various embodiments, facilitating daily ingestion of the silver compound including the silver particles 100 prior to chemotherapy may include ingesting the silver compound including the silver particles 100 any suitable number of times a day. For example, ingesting an amount of the silver compound including the silver particles 100 can include ingesting the silver compound once a day, two times a day, three times a day, etc. prior to receiving chemotherapy, among other quantities that are possible, each of which is contemplated herein.

In at least some embodiments in which the silver compound including the silver particles 100 is ingested at least twice daily, each ingestion is separated by a predetermined minimum amount of time, which can be any suitable amount of time that can facilitating protecting the blood cells of the subject. Example predetermined minimum amounts of time can include, but are not limited to, one hour, two hours, four hours, six hours, eight hours, twelve hours, etc., among other minimum amounts of time that are possible, each of which is contemplated herein.

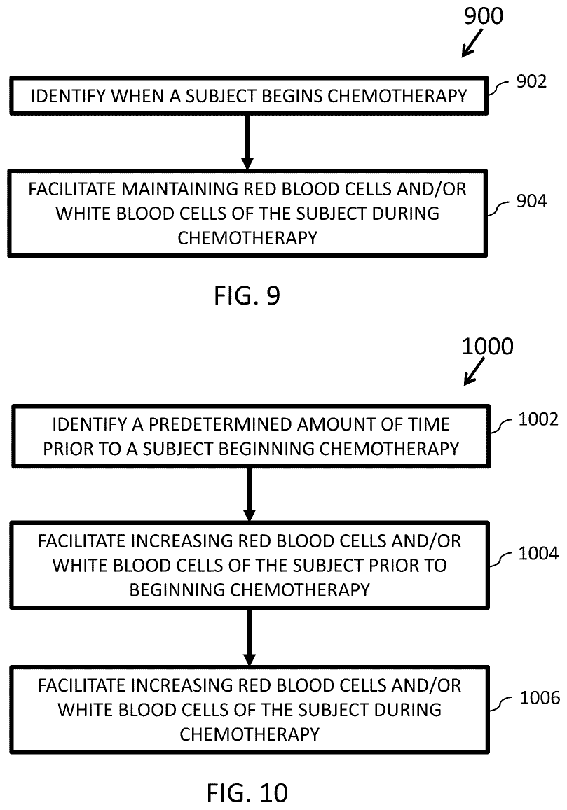

With reference to FIG. 9, FIG. 9 is a block diagram of one embodiment of a method 900 maintaining the red blood cells (e.g., red blood cell count) and/or white blood cells (e.g., white blood cell count) during chemotherapy. At least in the illustrated embodiment, the method 900 can begin by identifying a time when a subject begins chemotherapy (block 902).

A subject begins chemotherapy when the subject is exposed to an initial dose of the chemical substances used in chemotherapy (e.g., a cytotoxic and/or other drug(s)). A subject ends chemotherapy when the subject is exposed to a final dose of the chemical substances used in chemotherapy and/or includes a time subsequent to the subject's exposure to the final dose of the chemical substances used in chemotherapy.

The method 900 further includes facilitating maintaining the red blood cells (e.g., maintain the red blood cell count) and/or white blood cells (e.g., maintain the white blood cell count) of the subject during chemotherapy (block 904). In various embodiments, a silver compound including any of the various embodiments of the silver particles 100 discussed herein can facilitate maintaining the red blood cells and/or white blood cells of the subject during chemotherapy.

In some embodiments, facilitating maintaining the red blood cells (e.g., increase the red blood cell count) and/or white blood cells (e.g., increase the white blood cell count) of the subject during chemotherapy includes providing a silver compound to the subject (see, e.g., block 502 discussed with reference to FIG. 5). The silver compound can be provided to the subject before and/or after the subject begins chemotherapy. Further, the silver compound can be provided to the subject in any suitable manner that is known or developed in the future, as discussed elsewhere herein.

The silver compound can include any embodiment of the silver particles 100 discuss elsewhere herein. That is, the silver particles 100 may include nano-silver particles 100, colloidal silver particles 100, ionic silver particles 100, solution silver particles 100, and/or suspension silver particles 100.

In some embodiments, the silver compound includes an amount of silver particles 100, which can include any amount of silver particles 100 in the range of about 0.5 ppm to about 100 ppm, as discussed elsewhere herein. In certain embodiments, the amount of silver particles 100 is 10 ppm or 32 ppm, among other amounts that are possible, each of which is contemplated herein.

In additional or alternative embodiments, facilitating maintaining the red blood cells (e.g., increase the red blood cell count) and/or white blood cells (e.g., increase the white blood cell count) of the subject during chemotherapy includes facilitating daily ingestion of the silver compound including the silver particles 100 by the subject during chemotherapy (see, e.g., block 504 discussed with reference to FIG. 5). The subject can begin daily ingestion of the silver compound including the silver particles 100 at any point in time while the subject is receiving chemotherapy.

In some embodiments, the subject begins daily ingestion of the silver compound including the silver particles 100 on the initial day of chemotherapy. In other embodiments, the subject begins daily ingestion of the silver compound including the silver particles 100 on any suitable day subsequent to the initial day of chemotherapy.

Facilitating daily ingestion of the compound including the silver particles 100 while receiving chemotherapy may include any suitable technique and/or method that encourages and/or causes a subject to ingest the silver compound including the silver particles 100 on a daily and/or other similar regular basis. Examples of facilitating daily ingestion of the compound including the silver particles 100 can include, but are not limited to, by prescription, via instructions, by an advertisement, via a study, by a written article (e.g., online, journal, text, etc.), and/or via a recommendation (e.g., by a manufacturer, a healthcare professional, etc.), etc., among other similar techniques and/or methods that are possible, each of which is contemplated herein.

The silver compound including the silver particles 100 may be ingested in any suitable manner that is known or developed in the future. In various embodiments, the silver compound including the silver particles 100 is ingested orally, nasally, and/or intravenously (IV), among other techniques that are possible, each of which is contemplated herein.

In various embodiments, providing and/or facilitating daily ingestion of the silver compound including the silver particles 100 is performed in response to the subject beginning chemotherapy. In additional or alternative embodiments, providing and/or facilitating daily ingestion of the silver compound including the silver particles 100 while receiving chemotherapy is performed in response to a desire to protect the subject's blood cells during chemotherapy.

Facilitating daily ingestion of the silver compound including the silver particles 100 while receiving chemotherapy, in various embodiments, may include facilitating daily ingestion of any suitable amount of the silver compound capable of protecting a subject's blood cells. In some embodiments, facilitating daily ingestion can include facilitating daily ingestion of an amount of the silver compound in the range of about 0.15 oz to about 15 oz of the silver compound, among other amounts and/or ranges of amounts that are less than 0.15 oz or greater than 15 oz that are possible, each of which is contemplated herein. In certain embodiments, facilitating daily ingestion can include facilitating daily ingestion of 8 oz or up to about 8 oz of the silver compound, among other amounts that are less than 8 oz or greater than 8 oz that are possible, each of which is contemplated herein.

In various embodiments, facilitating daily ingestion of the silver compound including the silver particles 100 prior to chemotherapy may include ingesting the silver compound including the silver particles 100 any suitable number of times a day. For example, ingesting an amount of the silver compound including the silver particles 100 can include ingesting the silver compound once a day, two times a day, three times a day, etc. prior to receiving chemotherapy, among other quantities that are possible, each of which is contemplated herein.

In at least some embodiments in which the silver compound including the silver particles 100 is ingested at least twice daily, each ingestion is separated by a predetermined minimum amount of time, which can be any suitable amount of time that can facilitating protecting the blood cells of the subject. Example predetermined minimum amounts of time can include, but are not limited to, one hour, two hours, four hours, six hours, eight hours, twelve hours, etc., among other minimum amounts of time that are possible, each of which is contemplated herein.

With reference to FIG. 10, FIG. 10 is a block diagram of one embodiment of a method 1000 for increasing the red blood cells (e.g., red blood cell count) and/or white blood cells (e.g., white blood cell count) prior to and during chemotherapy. At least in the illustrated embodiment, the method 1000 can begin by identifying a predetermined amount of time prior to a subject beginning chemotherapy (block 1002).

The predetermined amount of time prior to the subject beginning chemotherapy can include any suitable amount of time prior to beginning chemotherapy that can provide sufficient time to increase the subject's red blood count and/or white blood cell count to a predetermined desired amount/level prior to beginning chemotherapy, which can include any suitable amount/level that is known or developed in the future. In various embodiments, the predetermined amount of time prior to the subject beginning chemotherapy is in the range of about one day to about sixty days prior to beginning chemotherapy, among other amounts of time and/or ranges of time that are possible, each of which is contemplated herein. In certain embodiments, the predetermined amount of time prior to the subject beginning chemotherapy is fourteen days, at least fourteen days, thirty days, or at least thirty days prior to beginning chemotherapy, among other amounts of time greater than thirty days and/or less than fourteen days that are possible, each of which is contemplated herein.

The method 1000 further includes facilitating increasing the red blood cells (e.g., increase the red blood cell count) and/or white blood cells (e.g., increase the white blood cell count) of the subject prior to the subject beginning chemotherapy (block 1004). The method 1000 also includes facilitating increasing the red blood cells (e.g., increase the red blood cell count) and/or white blood cells (e.g., increase the white blood cell count) of the subject during chemotherapy (block 1006). In some embodiments, increasing the red blood cells and/or white blood cells of the subject during chemotherapy includes further increasing the red blood cells (e.g., further increasing the red blood cell count) and/or further increasing white blood cells (e.g., further increasing the white blood cell count) of the subject compared to the increase in red blood cells and/or the increase in white blood cells achieved by the subject prior to beginning chemotherapy.

In some embodiments, facilitating increasing the red blood cells and/or white blood cells of the subject prior to and during chemotherapy includes providing a silver compound including silver particles 100 to the subject (see, e.g., block 402 discussed with reference to FIG. 4 and/or block 502 discussed with reference to FIG. 5). The silver compound can be provided to the subject in any suitable manner that is known or developed in the future. Examples of providing the silver compound to the user can include, but is not limited to, by prescription, over the counter, an online purchase, and/or a subscription, etc., among other techniques/methods that are possible, each of which is contemplated herein.

The silver compound can include any embodiment of the silver particles 100 discuss elsewhere herein. That is, the silver particles 100 may include nano-silver particles 100, colloidal silver particles 100, ionic silver particles 100, solution silver particles 100, and/or suspension silver particles 100.

In some embodiments, the silver compound includes an amount of silver particles 100, which can include any amount of silver particles 100 in the range of about 0.5 ppm to about 100 ppm, as discussed elsewhere herein. In certain embodiments, the amount of silver particles 100 is 10 ppm or 32 ppm, among other amounts that are possible, each of which is contemplated herein.

In additional or alternative embodiments, facilitating increasing the red blood cells and/or white blood cells of the subject prior to and during chemotherapy includes facilitating daily ingestion of the silver compound including the silver particles 100 by the subject prior to and during chemotherapy (see, e.g., block 404 discussed with reference to FIG. 4 and/or block 504 discussed with reference to FIG. 5).

The subject can begin daily ingestion of the silver compound including the silver particles 100 for any suitable period of time prior to beginning chemotherapy that can increase the subject's red blood count and/or white blood cell count to a predetermined desired amount/level prior to beginning chemotherapy, which can include any suitable amount/level that is known or developed in the future. In various embodiments, the subject begins daily ingestion of the silver compound including the silver particles 100 in the range of about one day to about sixty days prior to beginning chemotherapy, among other amounts of time and/or ranges of time that are possible, each of which is contemplated herein. In certain embodiments, the subject begins daily ingestion of the silver compound including the silver particles 100 fourteen days, at least fourteen days, thirty days, or at least thirty days prior to beginning chemotherapy, among other amounts of time greater than thirty days and/or less than fourteen days that are possible, each of which is contemplated herein. The subject can continue daily ingestion of the silver compound including silver particles 100 during chemotherapy.

Facilitating daily ingestion of the compound including the silver particles 100 may include any suitable technique and/or method that encourages and/or causes a subject to ingest the silver compound including the silver particles 100 on a daily and/or other similar regular basis. Examples of facilitating daily ingestion of the compound including the silver particles 100 can include, but are not limited to, by prescription, via instructions, by an advertisement, via a study, by a written article (e.g., online, journal, text, etc.), and/or via a recommendation (e.g., by a manufacturer, a healthcare professional, etc.), etc., among other similar techniques and/or methods that are possible, each of which is contemplated herein.

The silver compound including the silver particles 100 may be ingested in any suitable manner that is known or developed in the future. In various embodiments, the silver compound including the silver particles 100 is ingested orally, nasally, and/or intravenously (IV), among other techniques that are possible, each of which is contemplated herein.

In various embodiments, providing and/or facilitating daily ingestion of the silver compound including the silver particles 100 is performed in response to the subject preparing to begin chemotherapy. In additional or alternative embodiments, providing and/or facilitating daily ingestion of the silver compound including the silver particles 100 prior to receiving chemotherapy is performed in response to a desire to protect the subject's blood cells during chemotherapy.

Facilitating daily ingestion of the silver compound including the silver particles 100, in various embodiments, may include facilitating daily ingestion of any suitable amount of the silver compound capable of protecting a subject's blood cells. In some embodiments, facilitating daily ingestion can include facilitating daily ingestion of an amount of the silver compound in the range of about 0.15 oz to about 15 oz of the silver compound, among other amounts and/or ranges of amounts that are less than 0.15 oz or greater than 15 oz that are possible, each of which is contemplated herein. In certain embodiments, facilitating daily ingestion can include facilitating daily ingestion of 8 oz or up to about 8 oz of the silver compound, among other amounts that are less than 8 oz or greater than 8 oz that are possible, each of which is contemplated herein.

In various embodiments, facilitating daily ingestion of the silver compound including the silver particles 100 may include ingesting the silver compound including the silver particles 100 any suitable number of times a day. For example, ingesting an amount of the silver compound including the silver particles 100 can include ingesting the silver compound once a day, two times a day, three times a day, etc., among other quantities that are possible, each of which is contemplated herein.

In at least some embodiments in which the silver compound including the silver particles 100 is ingested at least twice daily, each ingestion is separated by a predetermined minimum amount of time, which can be any suitable amount of time that can facilitating protecting the blood cells of the subject. Example predetermined minimum amounts of time can include, but are not limited to, one hour, two hours, four hours, six hours, eight hours, twelve hours, etc., among other minimum amounts of time that are possible, each of which is contemplated herein.

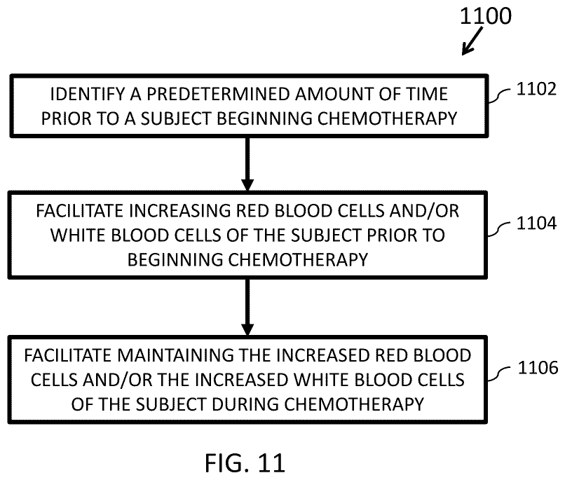

With reference to FIG. 11, FIG. 11 is a block diagram of one embodiment of a method 1100 for increasing the red blood cells (e.g., red blood cell count) and/or white blood cells (e.g., white blood cell count) of a subject prior to chemotherapy and maintaining the increased red blood cells (e.g., the increased red blood cell count) and/or the increased white blood cells (e.g., the increased white blood cell count) during chemotherapy. At least in the illustrated embodiment, the method 1100 can begin by identifying a predetermined amount of time prior to a subject beginning chemotherapy (block 1102).

The predetermined amount of time prior to the subject beginning chemotherapy can include any suitable amount of time prior to beginning chemotherapy that can provide sufficient time to increase the subject's red blood count and/or white blood cell count to a predetermined desired amount/level prior to beginning chemotherapy, which can include any suitable amount/level that is known or developed in the future. In various embodiments, the predetermined amount of time prior to the subject beginning chemotherapy is in the range of about one day to about sixty days prior to beginning chemotherapy, among other amounts of time and/or ranges of time that are possible, each of which is contemplated herein. In certain embodiments, the predetermined amount of time prior to the subject beginning chemotherapy is fourteen days, at least fourteen days, thirty days, or at least thirty days prior to beginning chemotherapy, among other amounts of time greater than thirty days and/or less than fourteen days that are possible, each of which is contemplated herein.

The method 1100 further includes facilitating increasing the red blood cells (e.g., increase the red blood cell count) and/or white blood cells (e.g., increase the white blood cell count) of the subject prior to the subject beginning chemotherapy (block 1104). The method 1100 also includes facilitating maintaining the increased red blood cells (e.g., the increased red blood cell count) and/or the increased white blood cells (e.g., the increased white blood cell count) of the subject during chemotherapy (block 1106).

In some embodiments, facilitating increasing the red blood cells and/or white blood cells of the subject prior to chemotherapy and maintaining the increased red blood cells and/or white blood cells during chemotherapy includes providing a silver compound including silver particles 100 to the subject (see, e.g., block 402 discussed with reference to FIG. 4 and/or block 502 discussed with reference to FIG. 5). The silver compound can be provided to the subject in any suitable manner that is known or developed in the future. Examples of providing the silver compound to the user can include, but is not limited to, by prescription, over the counter, an online purchase, and/or a subscription, etc., among other techniques/methods that are possible, each of which is contemplated herein.

The silver compound can include any embodiment of the silver particles 100 discuss elsewhere herein. That is, the silver particles 100 may include nano-silver particles 100, colloidal silver particles 100, ionic silver particles 100, solution silver particles 100, and/or suspension silver particles 100.

In some embodiments, the silver compound includes an amount of silver particles 100, which can include any amount of silver particles 100 in the range of about 0.5 ppm to about 100 ppm, as discussed elsewhere herein. In certain embodiments, the amount of silver particles 100 is 10 ppm or 32 ppm, among other amounts that are possible, each of which is contemplated herein.

In additional or alternative embodiments, facilitating increasing the red blood cells and/or white blood cells of the subject prior to chemotherapy and maintaining the increased red blood cells and/or white blood cells includes facilitating daily ingestion of the silver compound including the silver particles 100 by the subject prior to and during chemotherapy (see, e.g., block 404 discussed with reference to FIG. 4 and/or block 504 discussed with reference to FIG. 5).

The subject can begin daily ingestion of the silver compound including the silver particles 100 for any suitable period of time prior to beginning chemotherapy that can increase the subject's red blood count and/or white blood cell count to a predetermined desired amount/level prior to beginning chemotherapy, which can include any suitable amount/level that is known or developed in the future. In various embodiments, the subject begins daily ingestion of the silver compound including the silver particles 100 in the range of about one day to about sixty days prior to beginning chemotherapy, among other amounts of time and/or ranges of time that are possible, each of which is contemplated herein. In certain embodiments, the subject begins daily ingestion of the silver compound including the silver particles 100 fourteen days, at least fourteen days, thirty days, or at least thirty days prior to beginning chemotherapy, among other amounts of time greater than thirty days and/or less than fourteen days that are possible, each of which is contemplated herein. The subject can continue daily ingestion of the silver compound including silver particles 100 during chemotherapy.

Facilitating daily ingestion of the compound including the silver particles 100 may include any suitable technique and/or method that encourages and/or causes a subject to ingest the silver compound including the silver particles 100 on a daily and/or other similar regular basis. Examples of facilitating daily ingestion of the compound including the silver particles 100 can include, but are not limited to, by prescription, via instructions, by an advertisement, via a study, by a written article (e.g., online, journal, text, etc.), and/or via a recommendation (e.g., by a manufacturer, a healthcare professional, etc.), etc., among other similar techniques and/or methods that are possible, each of which is contemplated herein.

The silver compound including the silver particles 100 may be ingested in any suitable manner that is known or developed in the future. In various embodiments, the silver compound including the silver particles 100 is ingested orally, nasally, and/or intravenously (IV), among other techniques that are possible, each of which is contemplated herein.

In various embodiments, providing and/or facilitating daily ingestion of the silver compound including the silver particles 100 is performed in response to the subject preparing to begin chemotherapy. In additional or alternative embodiments, providing and/or facilitating daily ingestion of the silver compound including the silver particles 100 prior to receiving chemotherapy is performed in response to a desire to protect the subject's blood cells during chemotherapy.

Facilitating daily ingestion of the silver compound including the silver particles 100, in various embodiments, may include facilitating daily ingestion of any suitable amount of the silver compound capable of protecting a subject's blood cells. In some embodiments, facilitating daily ingestion can include facilitating daily ingestion of an amount of the silver compound in the range of about 0.15 oz to about 15 oz of the silver compound, among other amounts and/or ranges of amounts that are less than 0.15 oz or greater than 15 oz that are possible, each of which is contemplated herein. In certain embodiments, facilitating daily ingestion can include facilitating daily ingestion of 8 oz or up to about 8 oz of the silver compound, among other amounts that are less than 8 oz or greater than 8 oz that are possible, each of which is contemplated herein.

In various embodiments, facilitating daily ingestion of the silver compound including the silver particles 100 may include ingesting the silver compound including the silver particles 100 any suitable number of times a day. For example, ingesting an amount of the silver compound including the silver particles 100 can include ingesting the silver compound once a day, two times a day, three times a day, etc., among other quantities that are possible, each of which is contemplated herein.

In at least some embodiments in which the silver compound including the silver particles 100 is ingested at least twice daily, each ingestion is separated by a predetermined minimum amount of time, which can be any suitable amount of time that can facilitating protecting the blood cells of the subject. Example predetermined minimum amounts of time can include, but are not limited to, one hour, two hours, four hours, six hours, eight hours, twelve hours, etc., among other minimum amounts of time that are possible, each of which is contemplated herein.

Various embodiments of methods 300, 400, 500, 600, 700, 800, 900, 1000, and 1100 can further include ingesting a supplemental amount of the silver particles 100 a predetermined amount of time prior to receiving a particular treatment of chemotherapy and/or each particular treatment of chemotherapy. The supplemental amount of the silver particles 100 can include any suitable amount of the silver particles 100 or any of the amounts of the silver particles 100 discussed herein.

Further, the predetermined amount of time may include a suitable time prior to receiving a treatment of chemotherapy. In various embodiments, the time for ingesting the supplemental amount of the silver particles 100 is in the range of about one hour to about three hours prior to a subject receiving a treatment of chemotherapy. In certain embodiments, the time for ingesting the supplemental amount of the silver particles 100 is two hours prior to a subject receiving a treatment of chemotherapy.

Aspects of the various embodiments discussed above may be embodied as a compound and/or method for using silver compounds to protect the blood cells of a subject. Reference throughout this specification to one embodiment, an embodiment, or similar language means that a particular feature, structure, or characteristic described in connection with the embodiment is included in at least one embodiment. Thus, appearances of the phrases in one embodiment, in an embodiment, and similar language throughout this specification may, but do not necessarily, all refer to the same embodiment, but mean one or more but not all embodiments unless expressly specified otherwise. The terms including, comprising, having, and variations thereof mean including but not limited to, unless expressly specified otherwise. An enumerated listing of items does not imply that any or all of the items are mutually exclusive, unless expressly specified otherwise. The terms, “a,” “an,” and “the,” also refer to one or more unless expressly specified otherwise.

In addition, as used herein, the term, “set,” can mean one or more, unless expressly specified otherwise. The term, “sets,” can mean multiples of or a plurality of one or mores, ones or more, and/or ones or mores consistent with set theory, unless expressly specified otherwise.

Furthermore, the described features, structures, or characteristics of the embodiments may be combined in any suitable manner. One skilled in the relevant art will recognize, however, that embodiments may be practiced without one or more of the specific details, or with other methods, components, materials, and so forth. In other instances, well-known structures, materials, or operations are not shown or described in detail to avoid obscuring aspects of an embodiment.

Aspects of the embodiments are described above with reference to schematic flowchart diagrams and/or schematic block diagrams of particles (e.g., silver particles 100) and methods according to various embodiments. It should also be noted that, in some alternative implementations, the functions noted in the block may occur out of the order noted in the Figures. For example, two blocks shown in succession may, in fact, be executed substantially concurrently, or the blocks may sometimes be executed in the reverse order, depending upon the functionality involved. Other steps and methods may be conceived that are equivalent in function, logic, or effect to one or more blocks, or portions thereof, of the illustrated Figures.

Although various arrow types and line types may be employed in the flowchart and/or block diagrams, they are understood not to limit the scope of the corresponding embodiments. Indeed, some arrows or other connectors may be used to indicate only the logical flow of the depicted embodiment. For instance, an arrow may indicate a waiting or monitoring period of unspecified duration between enumerated steps of the depicted embodiment.

The description of elements in each figure may refer to elements of proceeding figures. Like numbers refer to like elements in all figures, including alternate embodiments of like elements.

The various disclosed embodiments may be practiced in other specific forms. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

1. A method, comprising:
providing a compound comprising silver (Ag) particles to a subject; and
facilitating daily ingestion of the compound by the subject,
wherein providing the compound to the subject and facilitating daily ingestion of the compound by the subject is performed to protect blood cells in the subject.

2. The method of claim 1, wherein the compound comprises 0.5 ppm to 100 ppm of the Ag particles.

3. The method of claim 2, wherein the Ag particles comprise one of nano-silver particles and colloidal silver particles.

4. The method of claim 2, wherein facilitating daily ingestion of the compound by the subject comprises facilitating daily ingestion of an amount of the compound in the range of 0.15 ounces to 15 ounces.

5. The method of claim 2, wherein facilitating daily ingestion of the compound by the subject comprises facilitating daily ingestion of an amount of the compound in the range of 0.15 ounces to 8 ounces.

6. The method of claim 2, wherein facilitating daily ingestion of the compound by the subject comprises facilitating daily ingestion of 8 ounces of the compound.

7. The method of claim 2, wherein facilitating daily ingestion of the compound by the subject comprises facilitating ingestion of the compound two times daily.

8. The method of claim 7, wherein facilitating ingestion of the compound two times daily comprises facilitating ingestion of 3 ounces or 4 ounces of the compound two times daily.

9. The method of claim 8, wherein the two times daily are separated by a predetermined minimum amount of time.

10. The method of claim 1, wherein providing the compound to the subject and facilitating ingestion of the compound by the subject is performed in response to the subject preparing for chemotherapy.

11. The method of claim 10, wherein providing the compound to the subject and facilitating ingestion of the compound by the subject is further performed in response to the subject currently receiving chemotherapy.

12. The method of claim 1, wherein providing the compound to the subject and facilitating ingestion of the compound by the subject is performed in response to the subject currently receiving chemotherapy.

13. The method of claim 1, wherein protecting the blood cells in the subject comprises at least one of raising and maintaining a count of blood cells in the subject.

14. The method of claim 13, wherein the count of blood cells in the subject comprises a count of white blood cells.

15. The method of claim 13, wherein the count of blood cells in the subject comprises a count of red blood cells.

16. The method of claim 13, wherein the count of blood cells in the subject comprises a first count of white blood cells and a second count of red blood cells.

17. The method of claim 1, wherein the Ag particles comprise one of nano-silver particles and colloidal silver particles.

18. The method of claim 1, wherein the compound comprises 10 ppm of the Ag particles.

19. The method of claim 1, wherein the compound comprises 32 ppm of the Ag particles.

20. The method of claim 1, wherein:
the compound comprises one of 10 ppm and 32 ppm of the Ag particles; and
the Ag particles comprise nano-silver particles.
